(19) United States
(12) Patent Application Publication
(10) Pub. No.: US 2015/0025518 A1
(43) Pub. Date: Jan. 22, 2015

(54) BLOOD VESSEL INSERTION-TYPE TREATMENT DEVICE

(71) Applicant: TERUMO KABUSHIKI KAISHA, Shibuya-ku (JP)

(72) Inventors: Risato KOBAYASHI, Hadano-shi (JP); Junichi KOBAYASHI, Fujinomiya-shi (JP); Ryota Sugimoto, Hiratsuka-shi (JP); Ichirou Hirahara, Setagaya-ku (JP)

(73) Assignee: TERUMO KABUSHIKI KAISHA, Tokyo (JP)

(21) Appl. No.: 14/506,871

(22) Filed: Oct. 6, 2014

Related U.S. Application Data
(63) Continuation of application No. PCT/JP2013/002277, filed on Apr. 2, 2013.

(30) Foreign Application Priority Data
Apr. 5, 2012 (JP) 2012-086852

(51) Int. Cl. A61N 7/02; A61B 8/12

(52) U.S. Cl. CPC A61N 7/022; A61B 8/12; A61N 2007/0052
USPC 606/30

(57) ABSTRACT

A blood vessel insertion-type treatment device and method of cauterizing biological tissues are disclosed. The blood vessel insertion-type treatment device having a first torque transmission body and a first ultrasonic generator. The first torque transmission body has a longitudinal shape and having a proximal end and a distal end. The first torque transmission body transmits a torque which is supplied to the proximal end and which pivotally rotates the first torque transmission body in a longitudinal direction of the longitudinal shape. The first ultrasonic generator is disposed in the first torque transmission body. The first ultrasonic generator radiates ultrasonic waves for cauterizing biological tissues, which are apart from the first ultrasonic generator by a predetermined distance.

### CROSS-REFERENCES TO RELATED APPLICATIONS

This application is a continuation of International Application No. PCT/JP2013/002277 filed on Apr. 2, 2013, and claims priority to Japanese Application No. 2012-086852 filed on Apr. 5, 2012, the entire content of both of which is incorporated herein by reference.

### TECHNICAL FIELD

The present disclosure relates to a blood vessel insertion-type treatment device, and for example, relates to a blood vessel insertion-type treatment device which can be inserted into a blood vessel and can perform cauterization on biological tissues around the blood vessel through the inside of the blood vessel.

### BACKGROUND DISCUSSION

In recent years, it is understood that abnormal renal artery sympathetic nerve activity can cause congestive heart failure, renal failure, hypertension, and other cardio-renal diseases. In addition, it is also known that these diseases are treated by removing a renal artery sympathetic nerve. In order to perform cauterization on the renal artery sympathetic nerve, a renal neuromodulation apparatus can be performed by inserting an electrode into a renal artery and applying a pulse output electric field to the renal artery exchange nerve (See U.S. Pat. No. 7,653,438).

In the cauterization of the renal artery sympathetic nerve which is performed by the renal neuromodulation apparatus disclosed in U.S. Pat. No. 7,653,438 using the pulse output electric field, current density in a blood vessel intima increases to the maximum. For this reason, heat generated in the blood vessel intima can increase such that the cauterization is performed on the entire vessel wall including the blood vessel intima. Consequently, side effects such as intimal thickening and thrombosis may occur.

### SUMMARY

In accordance with an exemplary embodiment, a blood vessel insertion-type treatment device is disclosed, which can suppress damage to a blood vessel, when cauterizing biological tissues around the blood vessel such as a renal artery sympathetic nerve around a renal artery.

In accordance with an exemplary embodiment, a blood vessel insertion-type treatment device according to the present disclosure can include a first torque transmission body that has a longitudinal shape whose both ends have a proximal end and a distal end (or insertion end), and that transmits a torque which is supplied to the proximal end and which pivotally rotates the first torque transmission body in a longitudinal direction of the longitudinal shape, and a first ultrasonic generator that is disposed in the first torque transmission body and radiates ultrasonic waves. The first ultrasonic generator can cauterize biological tissues which are apart from the first ultrasonic generator by a predetermined distance.

In accordance with an exemplary embodiment, the biological tissues can be cauterized by using the ultrasonic waves radiated by the first ultrasonic generator. Therefore, damage to blood vessels interposed between the first ultrasonic generator and cauterizing target tissues can be relatively suppressed. In addition, an orientation of the first ultrasonic generator disposed in the first torque transmission body can be changed, by pivotally rotating the first torque transmission body in the longitudinal direction of the first torque transmission body. Therefore, without being limited to one specific point, the biological tissues around the blood vessels even when an ultrasonic generator is used can be cauterized.

In accordance with an exemplary embodiment, a blood vessel insertion-type treatment device of the present disclosure which is configured as described above, damage to blood vessels when biological tissues around the blood vessels are removed can be relatively suppressed.

In accordance with an exemplary embodiment, a blood vessel insertion-type treatment device is disclosed comprising: a first torque transmission body having a longitudinal shape with a proximal end and a distal end, and that transmits a torque which is supplied to the proximal end of the first torque transmission body and which pivotally rotates the first torque transmission body in a longitudinal direction of the longitudinal shape; a first ultrasonic generator disposed in the first torque transmission body, which radiates ultrasonic waves; and wherein the first ultrasonic generator cauterizes biological tissues which are apart from the first ultrasonic generator by a predetermined distance.

In accordance with an exemplary embodiment, a method of cauterizing biological tissues is disclosed, comprising: inserting a blood vessel insertion-type treatment device into a blood vessel, the blood vessel insertion-type treatment device comprising: a first torque transmission body having a longitudinal shape with a proximal end and a distal end; and a first ultrasonic generator disposed in the first torque transmission body, which radiates ultrasonic waves; supplying a torque to the proximal end of the first torque transmission body and which pivotally rotates the first torque transmission body in a longitudinal direction of the longitudinal shape; and cauterizing the biological tissues which are apart from the first ultrasonic generator by a predetermined distance with the first ultrasonic generator.

### BRIEF DESCRIPTION OF DRAWINGS

FIG. 1 is a view for illustrating a manual technique for removing a renal artery sympathetic nerve by using a blood vessel insertion-type treatment device according to a first exemplary embodiment of the present disclosure.

FIG. 2 is an enlarged view illustrating the vicinity of a renal artery into which a guiding catheter is inserted in FIG. 1.

FIG. 3 is a cross-sectional view taken along a longitudinal direction near a distal end of the blood vessel insertion-type treatment device according to the first exemplary embodiment.

FIG. 4 is a cross-sectional view taken along the longitudinal direction near a distal end of a blood vessel insertion-type treatment device according to a second exemplary embodiment.

FIG. 5 is a cross-sectional view taken along the longitudinal direction near a distal end of a blood vessel insertion-type treatment device according to a third exemplary embodiment.

FIG. 6 is a cross-sectional view taken along the longitudinal direction near a distal end of a blood vessel insertion-type treatment device according to a fourth exemplary embodiment.

FIG. 7 is a cross-sectional view taken along the longitudinal direction near a distal end of a blood vessel insertion-type treatment device according to a fifth exemplary embodiment.

FIG. 8 is a block diagram schematically illustrating an internal configuration of a transmission body drive unit.

FIG. 9 is a timing chart for illustrating principles in which ultrasonic waves can converge by a first ultrasonic generator according to the fifth exemplary embodiment.

FIG. 10 is a cross-sectional view taken along the longitudinal direction near a distal end of a blood vessel insertion-type treatment device according to a sixth exemplary embodiment.

FIG. 11 is a cross-sectional view taken along the longitudinal direction near a distal end of a blood vessel insertion-type treatment device according to a seventh exemplary embodiment.

FIG. 12 is a cross-sectional view taken along line XII-XII in FIG. 11.

FIG. 13 is a view illustrating a first modification example of a mesh balloon.

FIG. 14 is a cross-sectional view taken along line IX-IX in FIG. 13.

FIG. 15 is a view illustrating a second modification example of the mesh balloon.

FIG. 16 is a cross-sectional view taken along line XI-XI in FIG. 15.

FIG. 17 is a cross-sectional view of a blood vessel insertion-type treatment device inside a blood vessel taken along a direction perpendicular to the longitudinal direction, which is prepared for illustrating a third modification example of the mesh balloon.

FIG. 18 is a cross-sectional view of a blood vessel insertion-type treatment device inside a blood vessel taken along a direction perpendicular to the longitudinal direction, which is prepared for illustrating a fourth modification example of the mesh balloon.

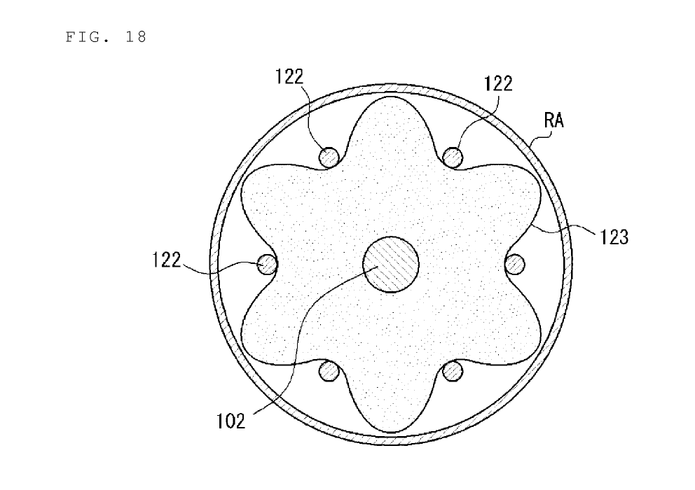

FIG. 19 is a cross-sectional view taken along the longitudinal direction near a distal end of a blood vessel insertion-type treatment device, which is prepared for illustrating a first modification example of a first ultrasonic transducer in the first embodiment.

FIG. 20 is a cross-sectional view taken along the longitudinal direction near a distal end of a blood vessel insertion-type treatment device, which is prepared for illustrating a first modification example of a first ultrasonic transducer in the fourth exemplary embodiment.

FIG. 21 is a cross-sectional view taken along a plane perpendicular to the longitudinal direction near a distal end of a blood vessel insertion-type treatment device, which is prepared for illustrating a modification example relating to an arrangement of the first ultrasonic generator and an imaging ultrasonic generator in the first exemplary embodiment.

FIG. 22 is a cross-sectional view taken along the longitudinal direction near the distal end of the blood vessel insertion-type treatment device, which is prepared for illustrating a second modification example of the first ultrasonic transducer and the imaging ultrasonic transducer in the first exemplary embodiment.

FIG. 23 is a cross-sectional view taken along the longitudinal direction near the distal end of the blood vessel insertion-type treatment device, which is prepared for illustrating a third modification example of the first ultrasonic transducer in the first exemplary embodiment.

FIG. 24 is a view illustrating a modification example of a first ultrasonic generator and a second ultrasonic generator in the fifth and sixth exemplary embodiments.

FIG. 25 is a view illustrating another modification example of the first ultrasonic generator in the fifth exemplary embodiment.

### DETAILED DESCRIPTION

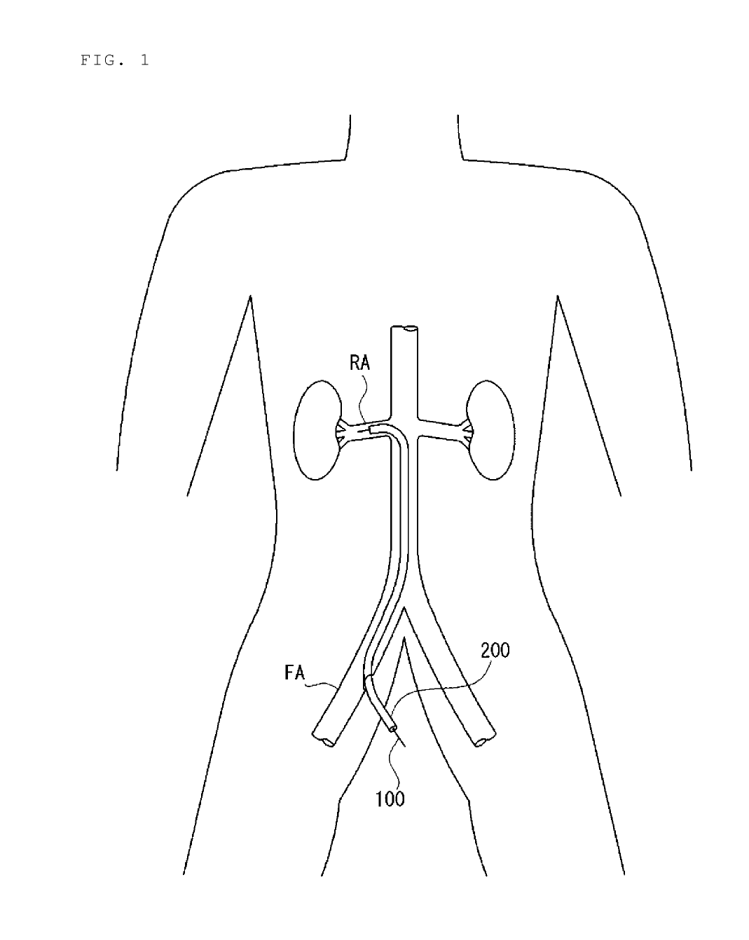

Hereinafter, embodiments of a blood vessel insertion-type treatment device to which the present disclosure is applied will be described with reference to the drawings. FIG. 1 is a view for illustrating a manual technique for removing a renal artery sympathetic nerve by using the blood vessel insertion-type treatment device according to a first exemplary embodiment of the present disclosure.

In order to apply a manual technique for removing the renal artery sympathetic nerve, a surgeon inserts a guiding catheter 200 into a femoral artery (“FA”) through a patient's thigh in advance, and causes a distal end of the guiding catheter 200 to reach a renal artery (“RA”).

A guide wire (not illustrated) can be used so that the guiding catheter 200 can reach the renal artery (RA).

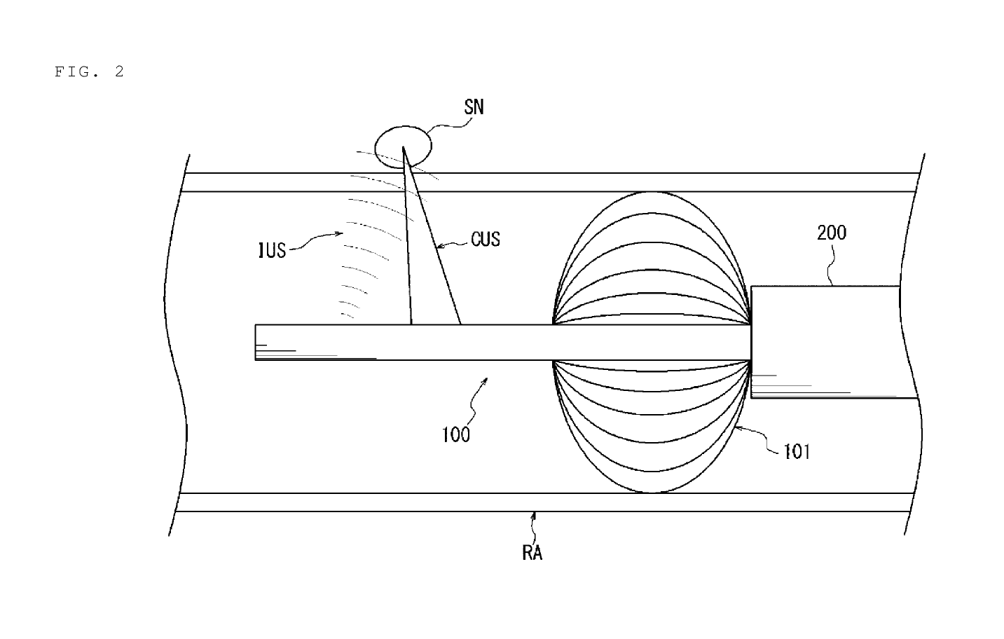

The guiding catheter 200 can have a tubular shape, and medical examination and treatment devices can be inserted into the guiding catheter 200. A blood vessel insertion-type treatment device 100 can have an entirely string shape, can have a distal end (or an insertion end) and a proximal end, and can be inserted into a lumen of the guiding catheter 200 through the distal end of the catheter 200. The surgeon inserts the blood vessel insertion-type treatment device 100 into the guiding catheter 200, and can cause the distal end of the treatment device 100 to protrude from the guiding catheter 200 (refer to FIG. 2). In a protruding state of the insertion end, a mesh balloon 101 disposed near the distal end of the blood vessel insertion-type treatment device 100 is expanded, thereby fixing the blood vessel insertion-type treatment device 100 into the renal artery (RA).

As disclosed below, the blood vessel insertion-type treatment device 100 can have an imaging function and a cauterizing function. In order to fulfill the imaging function, the blood vessel insertion-type treatment device 100 can radiate imaging ultrasonic waves (refer to reference sign IUS in FIG. 2). The surgeon causes the inserted blood vessel insertion-type treatment device 100 to fulfill the imaging function, thereby acquiring an image around the renal artery from the inside of the renal artery (RA).

Based on the acquired image, the surgeon can determine a sympathetic nerve (SN) to be cauterized, and can adjust a position of the blood vessel insertion-type treatment device 100 so that cauterizing ultrasonic waves are radiated to the determined sympathetic nerve (SN) (refer to the reference sign CUS in FIG. 2). After adjusting the position, the surgeon can cause the blood vessel insertion-type treatment device 100 to fulfill the cauterizing function, and cauterizes a desired sympathetic nerve.

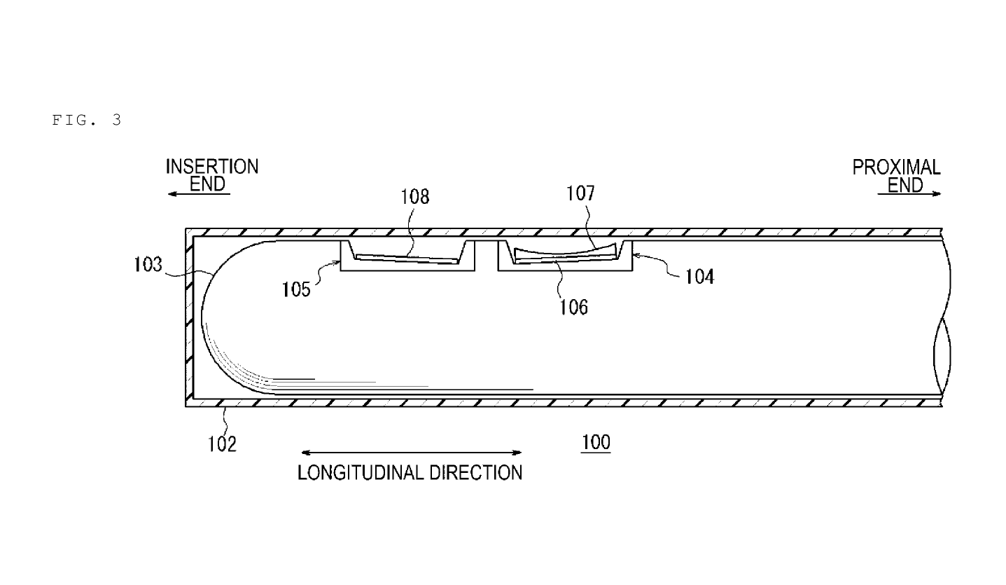

Next, a configuration of the blood vessel insertion-type treatment device 100 will be described with reference to FIG. 3. The blood vessel insertion-type treatment device 100 can be configured to include a sheath 102, a first torque transmission body 103, a first ultrasonic generator 104, an image acquisition unit 105, and a mesh balloon 101 (refer to FIG. 2).

The sheath 102 is formed in a tubular shape by a member having acoustic characteristics and flexibility. An end portion on the distal end side of the sheath 102 is open. In addition, when the sheath 102 starts to be used, the sheath 102 can be internally filled with a medium having acoustic transmission characteristics from the proximal end side.

The first torque transmission body 103 can be formed of a flexible member so as to extend from the proximal end to the distal end of the sheath 102. In a state where the distal end of the first torque transmission body 103 reaches the distal end of the sheath 102, the proximal end of the first torque transmission body 103 can protrude from the proximal end of the sheath 102.

An outer diameter of the first torque transmission body 103 is set to be narrower than an inner diameter of the sheath 102, and the first torque transmission body 103 can be pivotally rotatable inside the sheath 102 in the longitudinal direction. Therefore, in the proximal end of the first torque transmission body 103, if a torque which pivotally rotates the proximal end in the longitudinal direction is supplied, the supplied torque is transmitted to the distal end of the first torque transmission body 103, and the first torque transmission body 103 is entirely rotated inside the sheath 102. In addition, the first torque transmission body 103 is freely displaced inside the sheath 102 along the longitudinal direction.

The first ultrasonic generator 104 is disposed near the distal end of the first torque transmission body 103. The first ultrasonic generator 104 has a single unit of a first ultrasonic transducer 106 and an acoustic lens 107.

The first ultrasonic transducer 106 can be arranged so as to be capable of radiating the ultrasonic waves in a direction perpendicular to the longitudinal direction of the first torque transmission body 103, or in a direction which is tilted from the perpendicular direction to the distal end side by a predetermined angle. The first ultrasonic transducer 106 radiates cauterizing ultrasonic waves CUS having a frequency suitable for cauterization.

Depending on the frequency, a distance for transmitting the ultrasonic waves and a calorific value in a converging position of the ultrasonic waves are determined. Therefore, the frequency of the cauterizing ultrasonic waves CUS can be predetermined, based on an approximate interval from the inside of the renal artery (RA) to the renal artery sympathetic nerve (SN) and the calorific value required for the cauterization of the sympathetic nerve (SN).

A signal line extending from the first ultrasonic transducer 106 to the proximal end is connected to a cauterization control unit. The cauterization control unit can supply a drive signal to the first ultrasonic transducer 106 so as to generate the cauterizing ultrasonic waves CUS at the above-described frequency.

The acoustic lens 107 is disposed on a surface of the first ultrasonic transducer 106. The acoustic lens 107 can cause the ultrasonic waves to converge on a focus away from the acoustic lens 107 by a predetermined distance, thereby maximizing heat energy near the converging position. The acoustic lens 107 is formed to have a predetermined focal length, based on the approximate distance from the inside of the renal artery to the renal artery sympathetic nerve.

The image acquisition unit 105 is disposed on the distal end side further than the first ultrasonic generator 104 of the first torque transmission body 103.

The image acquisition unit 105 has a single unit of an imaging ultrasonic transducer 108.

The imaging ultrasonic transducer 108 can be arranged so as to be capable of radiating the ultrasonic waves in a direction perpendicular to the longitudinal direction of the first torque transmission body 103, or in a direction which is tilted from the perpendicular direction to the proximal end side by a predetermined angle. The imaging ultrasonic waves (IUS) suitable for acquisition of an image can be generated from the imaging ultrasonic transducer 108. In addition, the imaging ultrasonic transducer 108 can generate a pixel signal corresponding to the reflection waves of the imaging ultrasonic waves (IUS).

Depending on the frequency, the reflection waves of the ultrasonic waves change the resolution. Based on the resolution required for confirmation and medical examination of the position of a specific sympathetic nerve, the frequency of the imaging ultrasonic waves (IUS) is predetermined.

A signal line extending from the imaging ultrasonic transducer 108 to the proximal end is connected to an imaging control unit. The imaging control unit can supply the imaging ultrasonic transducer 108 with a drive signal so as to generate the imaging ultrasonic waves (IUS) at the above-described frequency.

In addition, the imaging control unit receives a pixel signal generated by the imaging ultrasonic transducer 108. The imaging control unit can create an image, based on an image signal corresponding to multiple locations from which the imaging ultrasonic waves are radiated. The radiation position of the imaging ultrasonic waves can be distinguishable by detecting a rotation position of the first torque transmission body 103 and a displacement position along the longitudinal direction, using an encoder or a position sensor. The radiation position is used in creating an image.

The mesh balloon 101 is disposed in the sheath 102.

A wire configuring the mesh balloon 101 is bent outward from the blood vessel insertion-type treatment device 100, and the wire is pressed against an inner wall of the blood vessel. In this manner, the blood vessel insertion-type treatment device 100 can be fixed into the blood vessel.

According to the blood vessel insertion-type treatment device 100 of the first exemplary embodiment which has the above-described configuration, heat energy at the converging position of the cauterizing ultrasonic waves can be maximized. Therefore, whereas the biological tissues distributed in a range from the inside of the blood vessel to the outside of the blood vessel can be cauterized, and damage to the blood vessel interposed between the biological tissues can be relatively suppressed. In accordance with an exemplary embodiment, in some cases, the first ultrasonic generator 104 can cauterize the biological tissues even when the acoustic lens 107 is not used.

In addition, according to the blood vessel insertion-type treatment device 100 of the first exemplary embodiment, the radiation position of the cauterizing ultrasonic waves (CUS) can be changed by using the first torque transmission body 103. In the cauterization of the biological tissues using the ultrasonic transducer, the ultrasonic waves can converge on a focus. Consequently, a region where the cauterization is possible is only in the vicinity of the focus. The blood vessel insertion-type treatment device 100 can be entirely rotated. However, it can be necessary to unfix the mesh balloon 101, thereby requiring complicated techniques.

Therefore, in the present exemplary embodiment, the first torque transmission body 103 can be used so as to change the radiation position. In this manner, the biological tissues distributed at various positions near the distal end of the sheath 102 can be cauterized. In accordance with an exemplary embodiment, the first torque transmission body 103 can be manually or automatically rotated.

In addition, according to the blood vessel insertion-type treatment device 100 of the first exemplary embodiment, the image acquisition unit 105 can be disposed near the first ultrasonic generator 104. Therefore, it can be relatively easy to confirm the biological tissues to be cauterized, and to confirm the cauterized state.

In addition, according to the blood vessel insertion-type treatment device 100 of the first exemplary embodiment, the distal end of the blood vessel insertion-type treatment device 100 can be temporarily fixed into the blood vessel by using the mesh balloon 101. In accordance with an exemplary embodiment, a blur in a reproduced image by fixing the blood vessel insertion-type treatment device 100 can be relatively reduced. In addition, a blur occurring at the radiation position of the cauterizing ultrasonic waves (CUS) can be relatively reduced. In addition, since the mesh balloon 101 is used, the blood flow can be relatively ensured. Accordingly, overheating of an inner wall portion of the blood vessel to which the cauterizing ultrasonic waves (CUS) are radiated can be relatively prevented, while the blood vessel insertion-type treatment device 100 can be fixed into the blood vessel.

In accordance with an exemplary embodiment, a blood vessel insertion-type treatment device according to a second exemplary embodiment will be disclosed. The second exemplary embodiment is different from the first embodiment in that the first ultrasonic generator can be used as the image acquisition unit. Hereinafter, the second exemplary embodiment focusing on points which are different from those in the first exemplary embodiment will be described. The same reference signs are given to elements having the function and configuration, which are the same as those in the first exemplary embodiment.

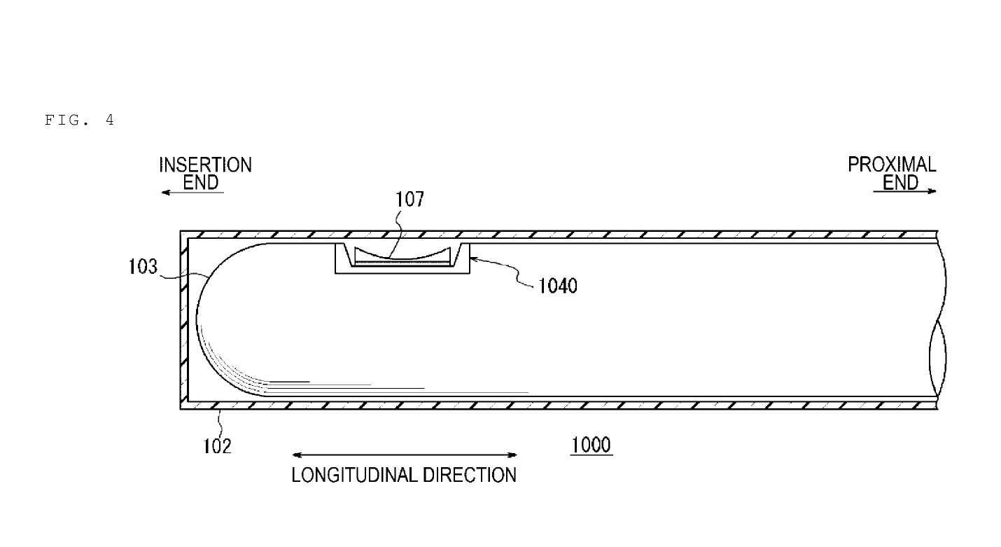

As illustrated in FIG. 4, a blood vessel insertion-type treatment device 1000 according to the second exemplary embodiment can be configured to include the sheath 102, the first torque transmission body 103, a first ultrasonic generator 1040, and the mesh balloon 101 (refer to FIG. 2). In the second exemplary embodiment, unlike the first exemplary embodiment, the image acquisition unit is not disposed. The second exemplary embodiment can have a configuration and a function of the sheath 102, a first torque transmission body 103, and the mesh balloon 101 which are the same as those in the first exemplary embodiment.

A configuration of a first ultrasonic generator 1040 is the same as that of the first embodiment. Similar to the first embodiment, the first ultrasonic generator 1040 generates the cauterizing ultrasonic waves (CUS). In addition, unlike the second exemplary embodiment, the first ultrasonic generator 1040 can generate the imaging ultrasonic waves (IUS). In addition, the first ultrasonic generator 1040 can generate a pixel signal in response to reflection waves of the imaging ultrasonic waves (IUS).

In accordance with an exemplary embodiment, various methods can be considered in order for the first ultrasonic generator 1040 to be allowed to have the above-described functions. For example, a configuration of generating the ultrasonic waves having the frequency applicable to both cauterizing and imaging can be employed, and a configuration of continuously switching between the frequency for the cauterizing and the frequency for the imaging.

The blood vessel insertion-type treatment device 1000 of the second exemplary embodiment which can be configured as described above can also suppress damage to the blood vessel interposed between the biological tissues, while being capable of cauterizing the biological tissues. In addition, the blood vessel insertion-type treatment device 1000 can also cauterize the biological tissues distributed at various positions near the distal end of the sheath 102. In addition, the blood vessel insertion-type treatment device 1000 can also reduce a blur on a reproduced image and a blur occurring at the radiation position of the cauterizing ultrasonic waves. In addition, the blood vessel insertion-type treatment device 1000 can also help prevent overheating of an inner wall portion of the blood vessel, while fixing the blood vessel insertion-type treatment device 1000 into the blood vessel.

In addition, according to the blood vessel insertion-type treatment device 1000 of the second embodiment, the first ultrasonic generator 1040 can be used in imaging. Therefore, it can be relatively easy to confirm the biological tissues to be cauterized, and to confirm the cauterized state. In addition, according to the blood vessel insertion-type treatment device 1000 of the second exemplary embodiment, it is no longer necessary to separately dispose the image acquisition unit, since the first ultrasonic generator 1040 can be used in fulfilling both the cauterizing function and the imaging function. Therefore, the manufacturing process can be simplified and the manufacturing costs can be reduced.

A blood vessel insertion-type treatment device according to a third exemplary embodiment of the present disclosure will be described. The third exemplary embodiment is different from the first exemplary embodiment in that the first ultrasonic generator and the image acquisition unit can be separately and pivotally rotated. Hereinafter, the third exemplary embodiment focusing on points which are different from those in the first exemplary embodiment will be described. In accordance with an exemplary embodiment, the same reference signs are given to elements having the function and configuration, which are the same as those in the first embodiment.

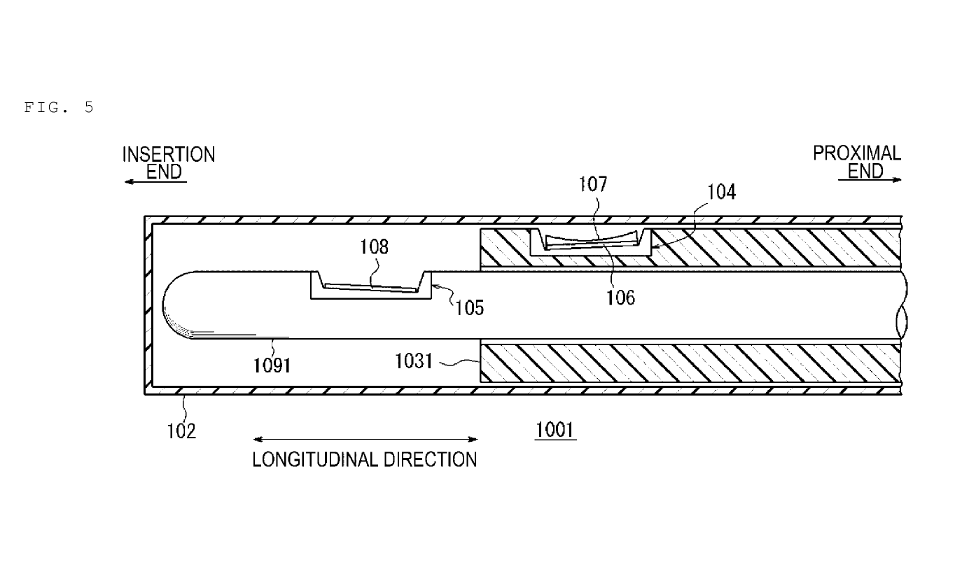

As illustrated in FIG. 5, a blood vessel insertion-type treatment device 1001 according to the third exemplary embodiment can be configured to include the sheath 102, a first torque transmission body 1031, a second torque transmission body 1091, the first ultrasonic generator 104, the image acquisition unit 105, and the mesh balloon 101 (refer to FIG. 2). A configuration and a function of the sheath 102, the first ultrasonic generator 104, and the mesh balloon 101 are the same as those in the first exemplary embodiment. In addition, configurations other than the arrangement of the image acquisition unit 105 are the same as those in the first exemplary embodiment.

The first torque transmission body 1031 can be formed of a flexible member in a tubular shape so as to extend from the proximal end to the distal end of the sheath 102. The other configurations and functions of the first torque transmission body 1031 are the same as those in the first embodiment. Therefore, the first torque transmission body 1031 can be pivotally rotated inside the sheath 102 in the longitudinal direction, and can be freely displaced in the longitudinal direction. In addition, the torque supplied to the proximal end of the first torque transmission body 1031 can be transmitted to the distal end, and thus, the first torque transmission body 1031 can be entirely rotated inside the sheath.

The second torque transmission body 1091 can be formed of a flexible member so as to extend from the proximal end to the distal end of the first torque transmission body 1031. In a state where the distal end of the second torque transmission body 1031 is protruded to the distal end of the first torque transmission body 1031, the proximal end of the second torque transmission body 1091 can be protrude from the proximal end of the first torque transmission body 1031.

The outer diameter of the second torque transmission body 1091 can be smaller than the inner diameter of the first torque transmission body 1031. The second torque transmission body 1091 can be pivotally rotated inside the first torque transmission body 1031 in the longitudinal direction. Therefore, in the proximal end of the second torque transmission body 1091, if a torque which pivotally rotates the proximal end in the longitudinal direction is supplied, the supplied torque is transmitted to the distal end of the second torque transmission body 1091, and the second torque transmission body 1091 can be entirely rotated inside the first torque transmission body 1031. In addition, the second torque transmission body 1091 can be freely displaced inside the first torque transmission body 1031 in the longitudinal direction.

The image acquisition unit 105 can be disposed near the distal end of the second torque transmission body 1091.

The blood vessel insertion-type treatment device 1001 of the third embodiment which is configured as described above can also help suppress damage to the blood vessel interposed between the biological tissues, while being capable of cauterizing the biological tissues. In addition, the blood vessel insertion-type treatment device 1001 can also cauterize the biological tissues distributed at various positions near the distal end of the sheath 102. In addition, the blood vessel insertion-type treatment device 1001 can also relatively easily confirm the biological tissues to be cauterized, and can confirm the cauterized state. In addition, the blood vessel insertion-type treatment device 1001 can also help reduce a blur on a reproduced image and a blur occurring at the radiation position of the cauterizing ultrasonic waves. In addition, the blood vessel insertion-type treatment device 1001 can also help prevent overheating of an inner wall portion of the blood vessel, while fixing the blood vessel insertion-type treatment device into the blood vessel.

According to the blood vessel insertion-type treatment device 1001 of the third embodiment, the first ultrasonic generator 104 and the image acquisition unit 105 can be separately and pivotally rotated, and/or can be separately displaced. Therefore, the first ultrasonic generator 104 and the image acquisition unit 105 can rotate at a speed suitable for the cauterizing and the imaging.

A blood vessel insertion-type treatment device according to a fourth exemplary embodiment of the present disclosure will be described. The fourth exemplary embodiment is different from the first exemplary embodiment in the configuration of the first ultrasonic generator. Hereinafter, the fourth exemplary embodiment focusing on points which are different from those in the first exemplary embodiment will be described. The same reference signs are given to elements having the function and configuration, which are the same as those in the first exemplary embodiment.

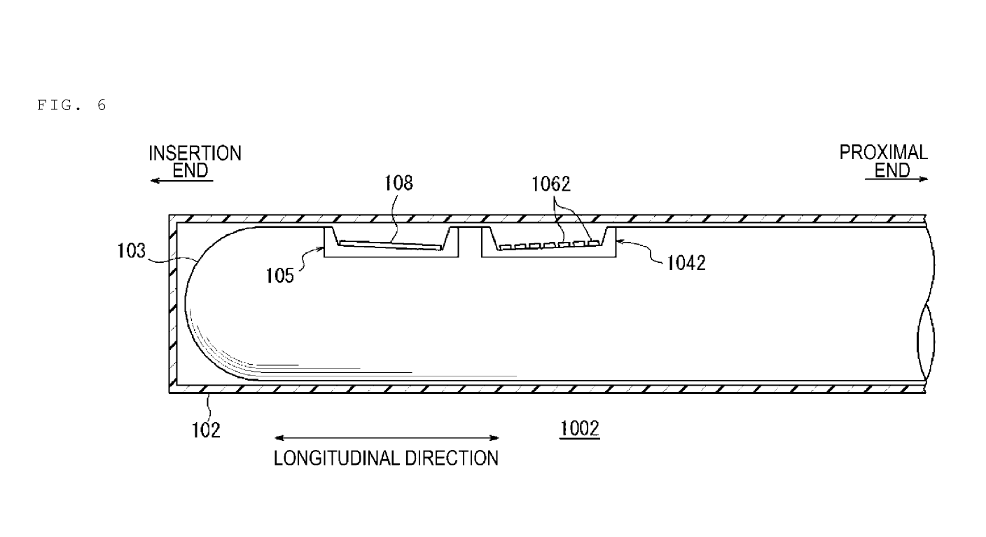

As illustrated in FIG. 6, a blood vessel insertion-type treatment device 1002 according to the fourth exemplary embodiment is configured to include the sheath 102, the first torque transmission body 103, a first ultrasonic generator 1042, the image acquisition unit 105, and the mesh balloon 101 (refer to FIG. 2). A configuration and a function of the sheath 102, the first torque transmission body 103, the image acquisition unit 105, and the mesh balloon 101 are the same as those in the first exemplary embodiment.

Unlike the first exemplary embodiment, the acoustic lens is not disposed in the first ultrasonic generator 1042, but a plurality or multiple first ultrasonic transducers 1062 can be arranged side by side along the longitudinal direction. The cauterization control unit can separately drive the first ultrasonic transducers 1062 so that a time point or a phase for radiating the ultrasonic waves is delayed from both ends to the center of the first ultrasonic generator 1042. Without using the acoustic lens, the ultrasonic waves can converge on the focus by driving the above-described first ultrasonic transducer 1062.

The blood vessel insertion-type treatment device 1002 of the fourth exemplary embodiment which is configured as described above can also help suppress damage to the blood vessel interposed between the biological tissues, while being capable of cauterizing the biological tissues. In addition, the blood vessel insertion-type treatment device 1002 can also cauterize the biological tissues distributed at various positions near the distal end of the sheath 102. In addition, the blood vessel insertion-type treatment device 1002 can also relatively easily confirm the biological tissues to be cauterized, and can confirm the cauterized state. In addition, the blood vessel insertion-type treatment device 1002 can also help reduce a blur on a reproduced image and a blur occurring at the radiation position of the cauterizing ultrasonic waves. In addition, the blood vessel insertion-type treatment device 1002 can also help prevent overheating of an inner wall portion of the blood vessel, while fixing the blood vessel insertion-type treatment device into the blood vessel.

In addition, according to the blood vessel insertion-type treatment device 1002 of the fourth exemplary embodiment, a focal length can be changed by adjusting a delay time such as the time point for generating the ultrasonic waves from the first ultrasonic transducer 1042. Therefore, according to the blood vessel insertion-type treatment device 1042, the biological tissues present in a wide range in terms of a distance from the blood vessel can be cauterized.

A blood vessel insertion-type treatment device according to a fifth exemplary embodiment of the present disclosure will be described. The fifth exemplary embodiment is different from the first exemplary embodiment in the configuration of the first ultrasonic generator. Hereinafter, the fifth exemplary embodiment focusing on points which are different from those in the first exemplary embodiment will be described. The same reference signs are given to elements having the function and configuration, which are the same as those in the first embodiment.

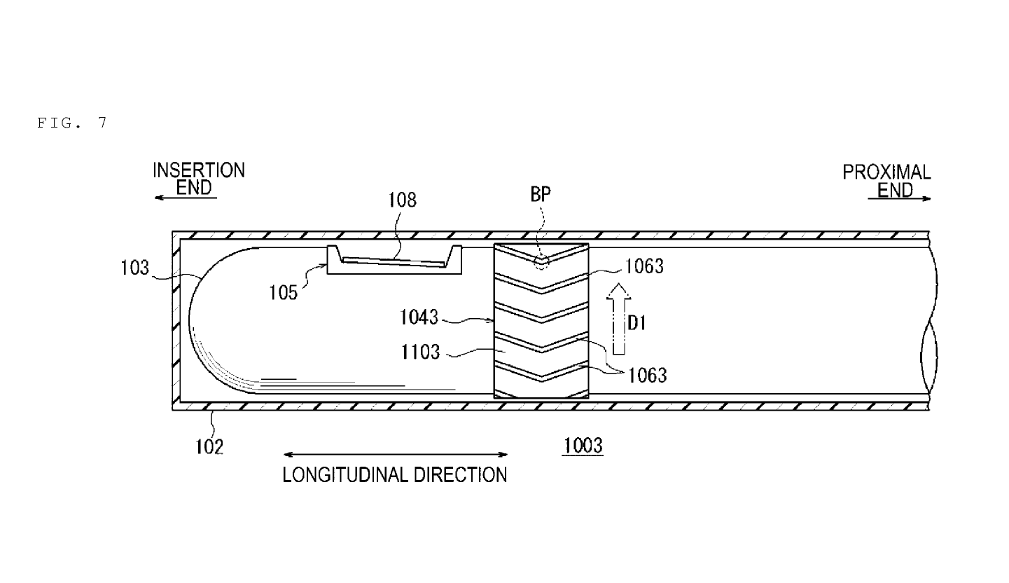

As illustrated in FIG. 7, a blood vessel insertion-type treatment device 1003 according to the fifth exemplary embodiment is configured to include the sheath 102, the first torque transmission body 103, a first ultrasonic generator 1043, the image acquisition unit 105, and the mesh balloon 101 (refer to FIG. 2). A configuration and a function of the sheath 102, the first torque transmission body 103, the image acquisition unit 105, and the mesh balloon 101 are the same as those in the first exemplary embodiment.

Unlike the first embodiment, the acoustic lens is not disposed in the first ultrasonic generator 1043. In addition, the first ultrasonic generator 1043 has a cylindrical main body 1103. In a state where the first torque transmission body 103 is inserted into the main body 1103, the main body 1103 can be fixed to the first torque transmission body 103.

In addition, multiple first ultrasonic transducers 1063 can be fixed to the first ultrasonic generator 1043 so as to be arranged side by side in a circumferential direction of the cylinder. The first ultrasonic transducer 1063 has a bent V-shape, and is arranged on the main body 1103 so that line segments bisecting bent portions (refer to reference numeral BP) are parallel to the circumferential direction, that is, perpendicular to the longitudinal direction of the blood vessel insertion-type treatment device 1003. Therefore, both bowl portions of the bent shape of the first ultrasonic transducer 1053 are arranged so as to tilt in the axial direction of the main body 1103. In accordance with an exemplary embodiment, the shape of the first ultrasonic transducer 1063 is not limited to the bent V-shape, and may be a curved shape such as a U-shape, which can allow the ultrasonic waves to converge on the focus.

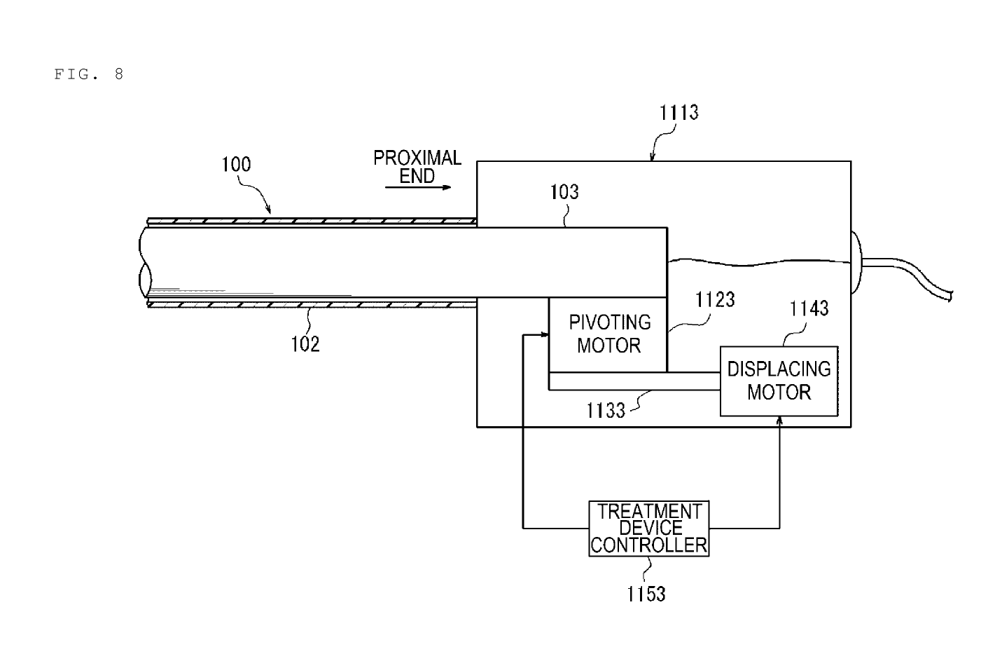

In addition, as illustrated in FIG. 8, the first torque transmission body 103 can be connected to a transmission body drive unit 1113 (pivot mechanism), on the proximal end side. The transmission body drive unit 1113 can be configured to include a pivoting motor 1123, a linear guide 1133, and a displacing motor 1143.

The pivoting motor 1123 can supply a torque which pivotally rotates the first torque transmission body 103 in the longitudinal direction. The linear guide 1133 fixes the pivoting motor 1123 at one end. In addition, the linear guide 1133 can be displaced along the longitudinal direction of the first torque transmission body 103. The displacing motor 1143 displaces the linear guide 1133 in the longitudinal direction.

The pivoting motor 1123 and the displacing motor 1143 can be driven based on a control of a treatment device controller 1153. The treatment device controller 1153 can control the pivoting motor 1123 so as to rotate the first ultrasonic generator 1043 in an opening direction (refer to reference numeral D1 in FIG. 7) of the bent portion BP of the first ultrasonic transducer 1063.

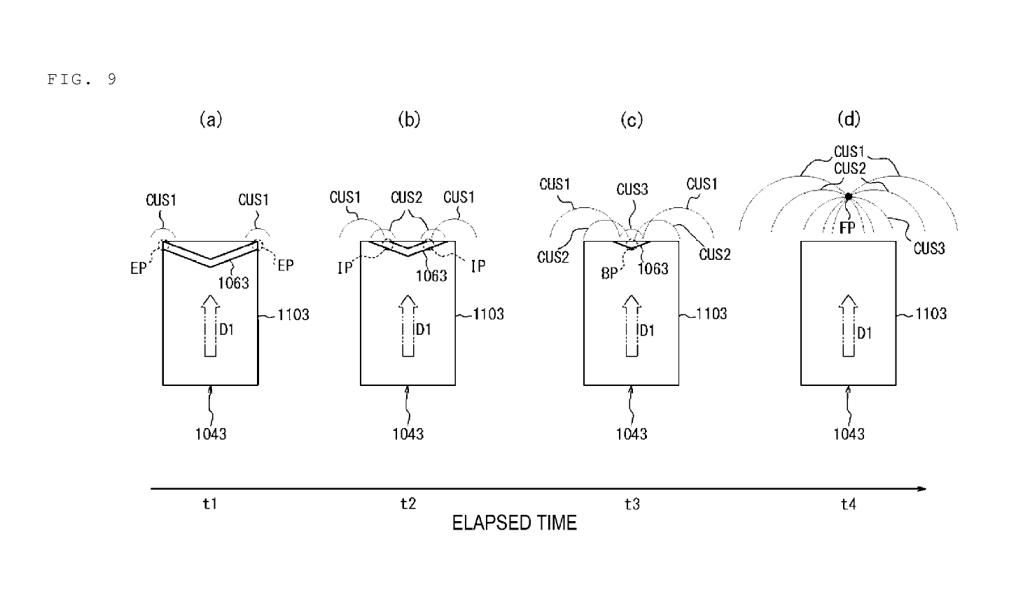

As described above, the ultrasonic waves can converge on the focus by driving the pivoting motor 1123, without using the acoustic lens as described below. The convergence of the ultrasonic waves according to the present embodiment will be described with reference to FIG. 9. In FIG. 9, in order to make description simpler, the convergence will be described using a single unit of the first ultrasonic transducer 1063.

The first ultrasonic generator 1043 is rotated so that a surface on an upper side in the drawing faces from bottom to top with the lapse of time (refer to reference numeral D1). At timing t1, when viewed from the paper surface side in FIG. 9, both end portions EP of the first ultrasonic transducer 1063 reach an upper end of the main body 1103, and the first ultrasonic transducer 1063 is driven so as to radiate ultrasonic waves CUS 1 (refer to FIG. 9(a)).

At timing t2, the rotation of the first ultrasonic generator 1043 causes a portion IP between both ends of the first ultrasonic transducer 1063 and the central bent portion to reach the upper end of the main body 1103, and the first ultrasonic transducer 1063 is driven again so as to radiate ultrasonic waves CUS 2 (refer to FIG. 9(b)). In addition, at timing t2, the ultrasonic waves CUS1 radiated at timing t1 are diffused.

Furthermore, at timing t3, the rotation of the first ultrasonic generator 1043 can cause the bent portion BP of the first ultrasonic transducer 1063 to reach the upper end of the main body 1103, and the first ultrasonic transducer 1063 is driven again so as to radiate ultrasonic waves CUS 3 (refer to FIG. 9(c)). In addition, at timing t3, the ultrasonic waves CUS1 and CUS2 radiated at timing t1 and t2 are diffused.

The ultrasonic waves CUS1, CUS2, and CUS3 radiated at timing t1, t2, and t3 interfere with one another (refer to an intersecting point of a wave front in FIG. 9), and an amplitude of the ultrasonic waves CUS1, CUS2, and CUS3 increases. At timing t4, the ultrasonic waves CUS1, CUS2, and CUS3 radiated at timing t1, t2, and t3 are all overlapped with one another on a focus FP (refer to FIG. 9(d)). In this manner, the ultrasonic waves can converge on the focus by rotating the first ultrasonic generator while causing the first ultrasonic generator 1063 having the configuration according to the present exemplary embodiment to generate the ultrasonic waves CUS, without using the acoustic lens.

The blood vessel insertion-type treatment device 1003 of the fifth exemplary embodiment which is configured as described above can also help suppress damage to the blood vessel interposed between the biological tissues, while being capable of cauterizing the biological tissues. In addition, the blood vessel insertion-type treatment device 1003 can also cauterize the biological tissues distributed at various positions near the distal end of the sheath 102. In addition, the blood vessel insertion-type treatment device 1003 can also relatively easily confirm the biological tissues to be cauterized, and can help confirm the cauterized state. In addition, the blood vessel insertion-type treatment device 1003 can also help reduce a blur on a reproduced image and a blur occurring at the radiation position of the cauterizing ultrasonic waves. In addition, the blood vessel insertion-type treatment device 1003 can also help prevent overheating of an inner wall portion of the blood vessel, while fixing the blood vessel insertion-type treatment device into the blood vessel.

In addition, according to the blood vessel insertion-type treatment device 1003 of the fifth exemplary embodiment, the focal length can be changed by adjusting cycles for causing the first ultrasonic transducer 1063 to generate the ultrasonic waves and by adjusting a rotation speed of the first torque transmission body 103. Therefore, according to the blood vessel insertion-type treatment device 1003, the biological tissues present in a wide range in terms of a distance from the blood vessel can be cauterized.

A blood vessel insertion-type treatment device according to a sixth exemplary embodiment of the present disclosure will be described. The sixth exemplary embodiment is different from the first exemplary embodiment in the configuration of the first ultrasonic generator. Hereinafter, the sixth exemplary embodiment focusing on points which are different from those in the first exemplary embodiment will be described. The same reference signs are given to elements having the function and configuration, which are the same as those in the first exemplary embodiment.

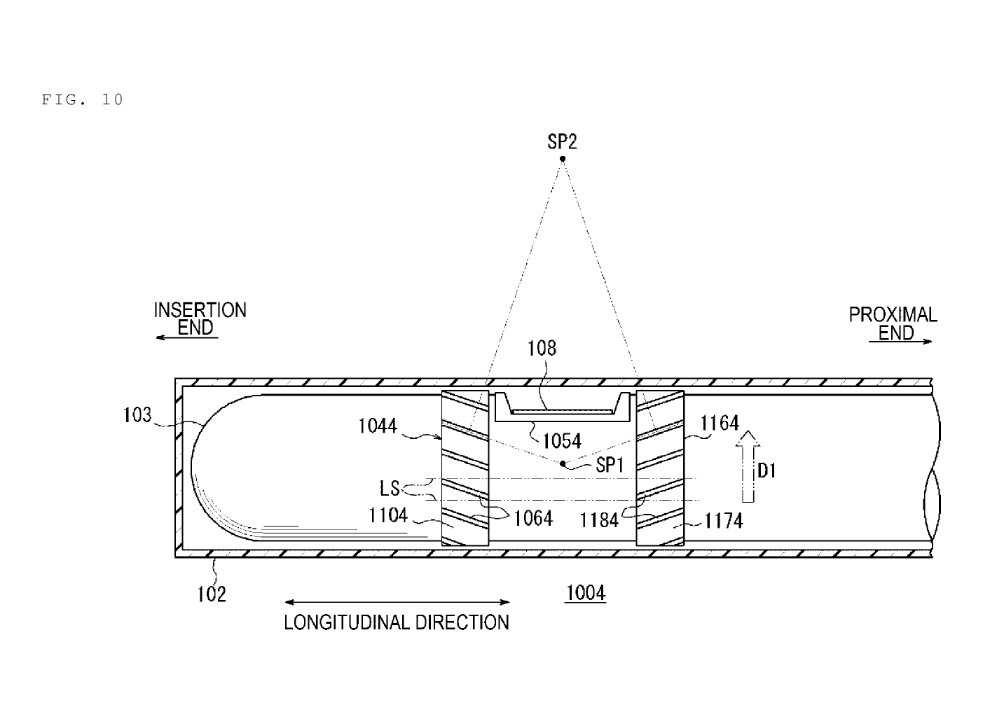

As illustrated in FIG. 10, a blood vessel insertion-type treatment device 1004 according to the sixth exemplary embodiment is configured to include the sheath 102, the first torque transmission body 103, a first ultrasonic generator 1044, a second ultrasonic generator 1164, an image acquisition unit 1054, and the mesh balloon 101. A configuration and a function of the sheath 102, the first torque transmission body 103, and the mesh balloon 101 are the same as those in the first exemplary embodiment.

Unlike the first exemplary embodiment, the acoustic lens is not disposed in the first ultrasonic generator 1044. In addition, the first ultrasonic generator 1044 has a cylindrical main body 1104. In a state where the first torque transmission body 103 is inserted into the main body 1104, the main body 1104 is fixed to the first torque transmission body 103. In addition, multiple first ultrasonic transducers 1064 can be fixed to the first ultrasonic generator 1044 so as to be arranged side by side in the circumferential direction of the cylinder. A first ultrasonic transducer 1064 can have a shape having a longitudinal direction, and is arranged so as to tilt to a rotation axis of the first torque transmission body 103.

The second ultrasonic generator 1164 can be fixed to the first torque transmission body 103 at a position interposed between the image acquisition unit 1054 and the first ultrasonic generator 1044 along the longitudinal direction of the first torque transmission body 103. The second ultrasonic generator 1164 can have a cylindrical main body 1174, similar to the first ultrasonic generator 1044, and can be fixed so that multiple second ultrasonic transducers 1184 are arranged side by side in the circumferential direction of the cylinder. The second ultrasonic transducers 1184 can also be arranged so as to tilt to the rotation axis of the first torque transmission body 103.

In accordance with an exemplary embodiment, the second ultrasonic transducers 1184 can be arranged so as to tilt to the rotation axis at the same angle in a direction opposite to the first ultrasonic transducer 1064, and so that both ends in the circumferential direction of the second ultrasonic transducers 1184 are overlapped with both ends of the first ultrasonic transducer 1064 in the circumferential direction (refer to line segments LS). Therefore, the first ultrasonic transducer 1064 and the second ultrasonic transducer 1184 are line-symmetric with respect to a line along the circumferential direction of the rotation made by the first torque transmission body 103.

In addition, the first torque transmission body 103 in the present exemplary embodiment is also connected to the transmission body drive unit 1113, similar to the fifth embodiment, and is controlled so that the first ultrasonic generator 1044 and the second ultrasonic generator 1164 are pivotally rotated in the longitudinal direction together with the first torque transmission body 103. In the present embodiment, the treatment device controller 1153 controls the pivoting motor 1123 so as to be rotated in a direction from an intersecting point (refer to reference numeral SP1) of extension lines taken along the longitudinal directions of the first ultrasonic transducer 1064 and the second ultrasonic transducer 1184 toward an intersecting point (refer to reference numeral SP2) of perpendicular lines to the longitudinal directions.

As disclosed above, the ultrasonic waves CUS can converge on the focus by driving the pivoting motor 1123, by utilizing the same principle described in the fifth exemplary embodiment, without using the acoustic lens.

The blood vessel insertion-type treatment device 1004 of the sixth exemplary embodiment which is configured as described above can also help suppress damage to the blood vessel interposed between the biological tissues, while being capable of cauterizing the biological tissues. In addition, the blood vessel insertion-type treatment device 1004 can also cauterize the biological tissues distributed at various positions near the distal end of the sheath 102. In addition, the blood vessel insertion-type treatment device 1004 can also relatively easily confirm the biological tissues to be cauterized, and can confirm the cauterized state. In addition, the blood vessel insertion-type treatment device 1004 can also help reduce a blur on a reproduced image and a blur occurring at the radiation position of the cauterizing ultrasonic waves. In addition, the blood vessel insertion-type treatment device 1004 can also help prevent overheating of an inner wall portion of the blood vessel, while fixing the blood vessel insertion-type treatment device into the blood vessel.

In addition, according to the blood vessel insertion-type treatment device 1004 of the sixth exemplary embodiment, the focal length can be changed by adjusting cycles for causing the first ultrasonic transducer 1064 and the second ultrasonic transducer 1184 to generate the ultrasonic waves and by adjusting a rotation speed of the first torque transmission body 103. Therefore, according to the blood vessel insertion-type treatment device 1004, the biological tissues present in a wide range in terms of a distance from the blood vessel can be cauterized.

In addition, according to the blood vessel insertion-type treatment device 1004 of the sixth exemplary embodiment, it can be relatively easy to manufacture an ultrasonic transducer used for the first ultrasonic transducer 1064 and the second ultrasonic transducer 1184. The ultrasonic transducer can be a piezoelectric element. Thus, it can be difficult to form the ultrasonic transducer in the bent shape as in the fifth exemplary embodiment. However, in the present exemplary embodiment, a linear-shaped piezoelectric element can be used. Accordingly, as compared to the ultrasonic transducer in the fifth exemplary embodiment, the manufacturing can be easily facilitated.

In addition, according to the blood vessel insertion-type treatment device 1004 of the sixth exemplary embodiment, the image acquisition unit 1054 is disposed at the position interposed between the first ultrasonic generator 1044 and the second ultrasonic generator 1164. Accordingly, even when the imaging ultrasonic waves IUS are radiated in a direction perpendicular to the first torque transmission body 103, the cauterizing ultrasonic waves CUS can be radiated onto the focus. Therefore, an image around the focus can be acquired without causing the imaging ultrasonic transducer 108 to be excessively tilted to the first torque transmission body 103. Whereas the blood vessel insertion-type treatment device 1004 needs to have a small diameter, it is difficult to arrange the imaging ultrasonic transducer 108 to be tilted. Therefore, according to the configuration of the present exemplary embodiment, the manufacturing is easily facilitated.

In accordance with an exemplary embodiment, a blood vessel insertion-type treatment device according to a seventh exemplary embodiment of the present disclosure will be described. The seventh exemplary embodiment is different from the first exemplary embodiment in the configuration of the first ultrasonic generator. Hereinafter, the seventh exemplary embodiment focusing on points which are different from those in the first exemplary embodiment will be described. Note that, the same reference signs are given to elements having the function and configuration which are the same as those in the first exemplary embodiment.

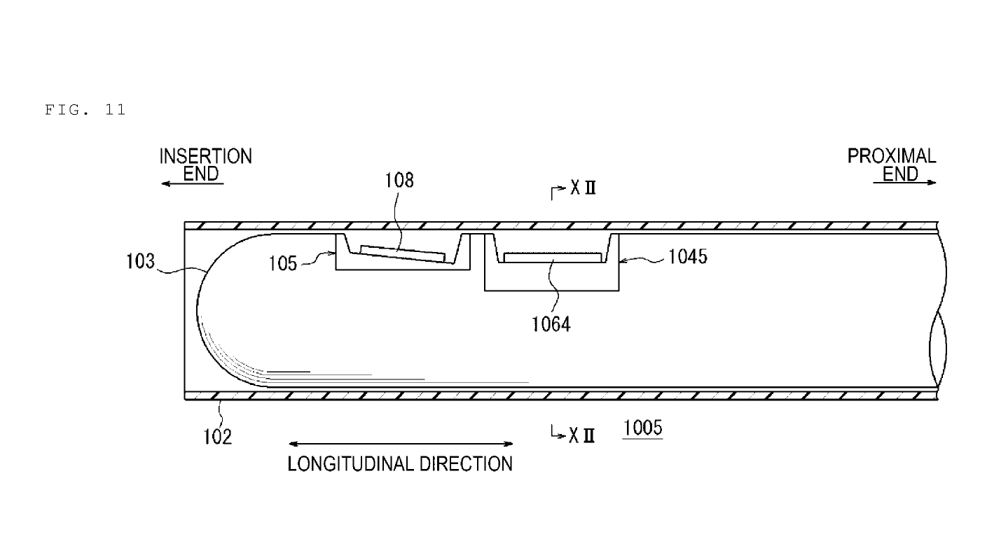

As illustrated in FIG. 11, a blood vessel insertion-type treatment device 1005 according to the seventh exemplary embodiment is configured to include the sheath 102, the first torque transmission body 103, a first ultrasonic generator 1045, the image acquisition unit 105, and the mesh balloon 101 (refer to FIG. 2). The seventh exemplary embodiment has a configuration and a function of the sheath 102, the first torque transmission body 103, the image acquisition unit 105, and the mesh balloon 101 which are the same as those in the first exemplary embodiment.

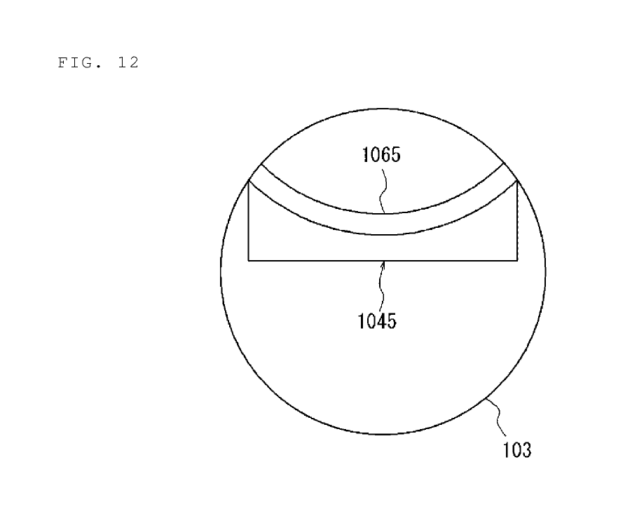

As illustrated in FIG. 11, the first ultrasonic generator 1045 has a single unit of a first ultrasonic transducer 1065. Unlike the first exemplary embodiment, the acoustic lens is not disposed in the first ultrasonic generator 1045. As illustrated in FIG. 12, the first ultrasonic transducer 1065 has a concave surface on a plane perpendicular to the longitudinal direction of the first torque transmission body 103. Since the first ultrasonic transducer 1065 has the concave surface on the plane perpendicular to the longitudinal direction, the ultrasonic waves radiated by the first ultrasonic transducer 1065 converge on the focus which is apart by a predetermined distance from the first ultrasonic transducer 1065 on the plane perpendicular to the longitudinal direction.

The blood vessel insertion-type treatment device 1005 of the seventh exemplary embodiment which is configured as described above can also suppress damage to the blood vessel interposed between the biological tissues, while being capable of cauterizing the biological tissues. In addition, the blood vessel insertion-type treatment device 1005 can also cauterize the biological tissues distributed at various positions near the distal end of the sheath 102. In addition, the blood vessel insertion-type treatment device 1005 can also help reduce a blur on a reproduced image and a blur occurring at the radiation position of the cauterizing ultrasonic waves. In addition, the blood vessel insertion-type treatment device 1005 can also help prevent overheating of an inner wall portion of the blood vessel, while fixing the blood vessel insertion-type treatment device 1005 into the blood vessel.

In addition, according to the blood vessel insertion-type treatment device 1005 of the seventh exemplary embodiment, it is not necessary to dispose the acoustic lens. Therefore, the manufacturing process can be simplified and the manufacturing costs can be reduced.

The present disclosure has been described with reference to the accompanying drawings and the embodiments. However, it should be noted that those skilled in the art can easily make various modifications or corrections based on the present disclosure. Therefore, all these modifications or corrections are intended to be included within the scope of the present disclosure.

For example, in the blood vessel insertion-type treatment devices 100, 1000, 1001, 1002, 1003, and 1004 according to the first to sixth exemplary embodiments, the mesh balloon 101 is disposed. However, a configuration may be made so that the blood vessel insertion-type treatment devices 100, 1000, 1001, 1002, 1003, and 1004 can be temporarily fixed into the blood vessel by using other balloons.

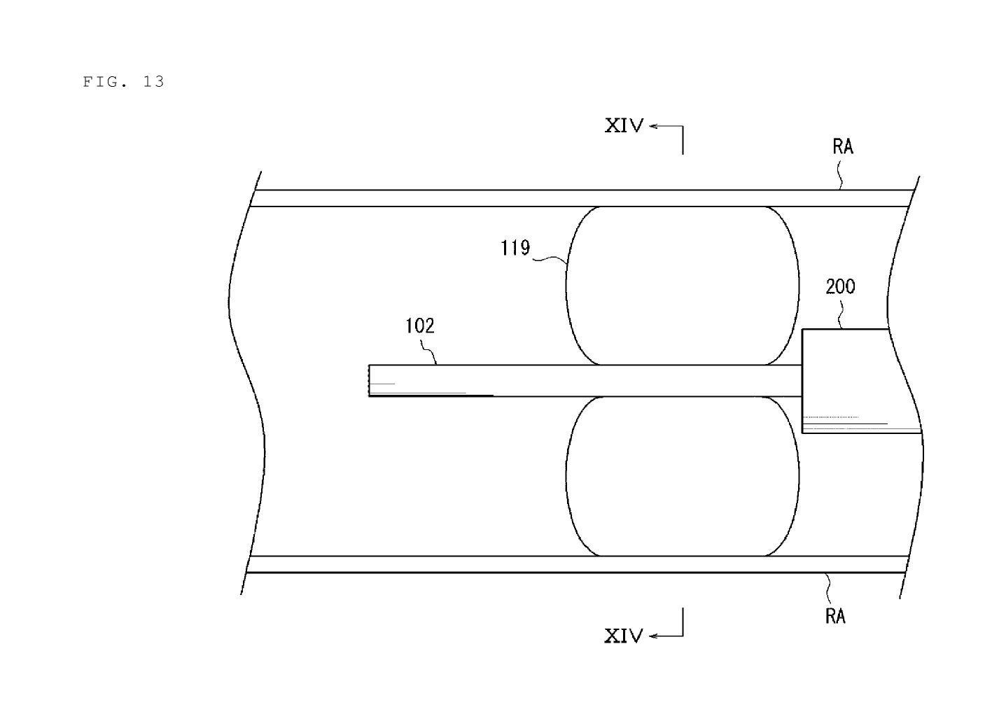

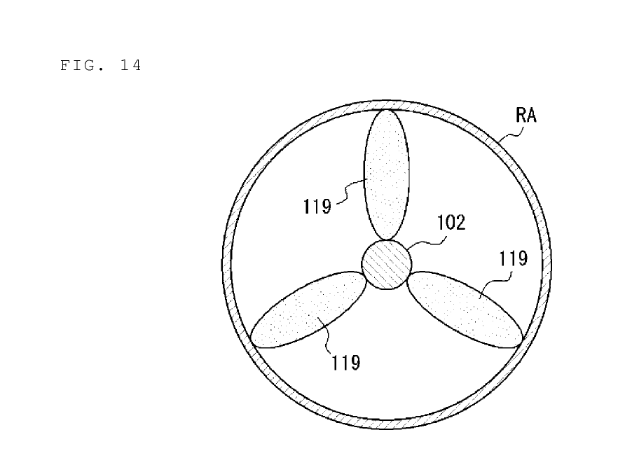

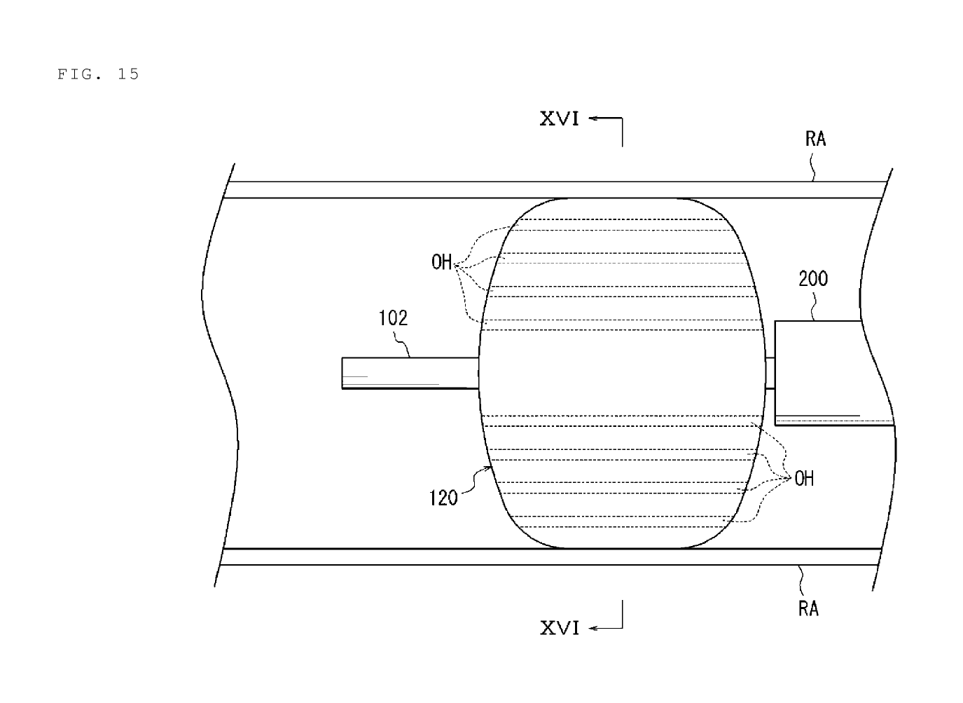

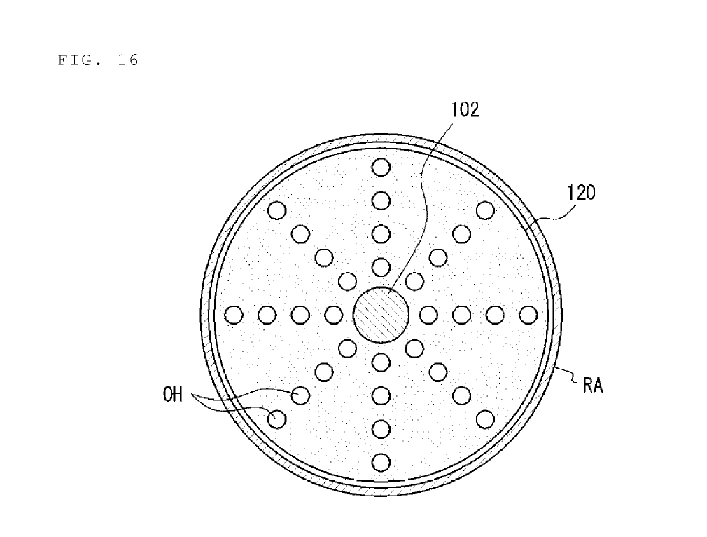

In accordance with an exemplary embodiment, it can be preferable to use a balloon to help prevent the overheating of the inner wall inside the blood vessel. For example, as illustrated in FIGS. 13 and 14, a configuration having multiple balloons 119 expandable in different directions around the sheath 102 can obtain an overheating prevention effect which is the same as that of the mesh balloon 101. In addition, for example, as illustrated in FIGS. 15 and 16, a configuration having a balloon 120 which is expandable to the entire circumference around the sheath 102 and in which a hole portion OH penetrating in the longitudinal direction is formed can also obtain the overheating prevention effect which is the same as that of the mesh balloon 101.

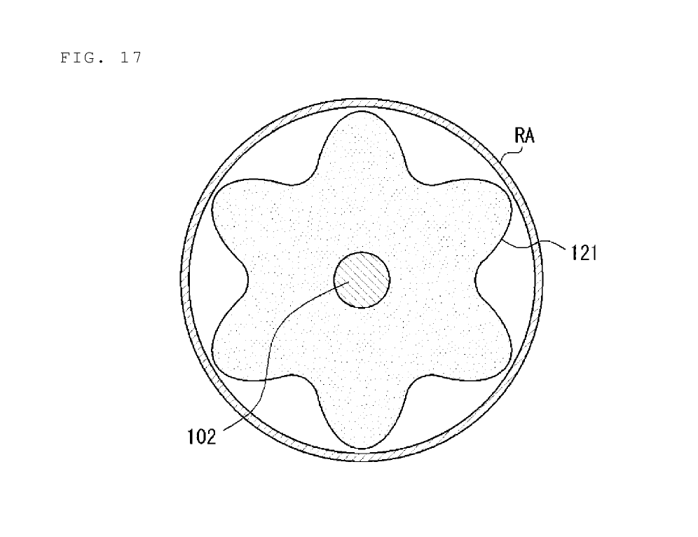

In addition, for example, as illustrated in FIG. 17, a configuration having a balloon 121 formed so that a cross-section taken along a plane perpendicular to the longitudinal direction has a star shape can also obtain the overheating prevention effect which is the same as that of the mesh balloon 101. In addition, for example, as illustrated in FIG. 16, a configuration in which a balloon 123 is partially expanded by using multiple wires 122 can also obtain the overheating prevention effect which is the same as that of the mesh balloon 101.

In accordance with an exemplary embodiment, a perfusion balloon and a cryo-balloon in which the inner wall of the blood vessel can be cooled by a refrigerant can be used. In the cauterization using the ultrasonic waves, the heating energy at the focus can be maximized. However, blood vessel walls including the inner wall of the blood vessel through which the ultrasonic waves are propagated prior to convergence may also be heated by the ultrasonic waves. Therefore, a possibility of damage which may occur on the inner wall of the blood vessel, by using the cooling-type balloon can be reduced.

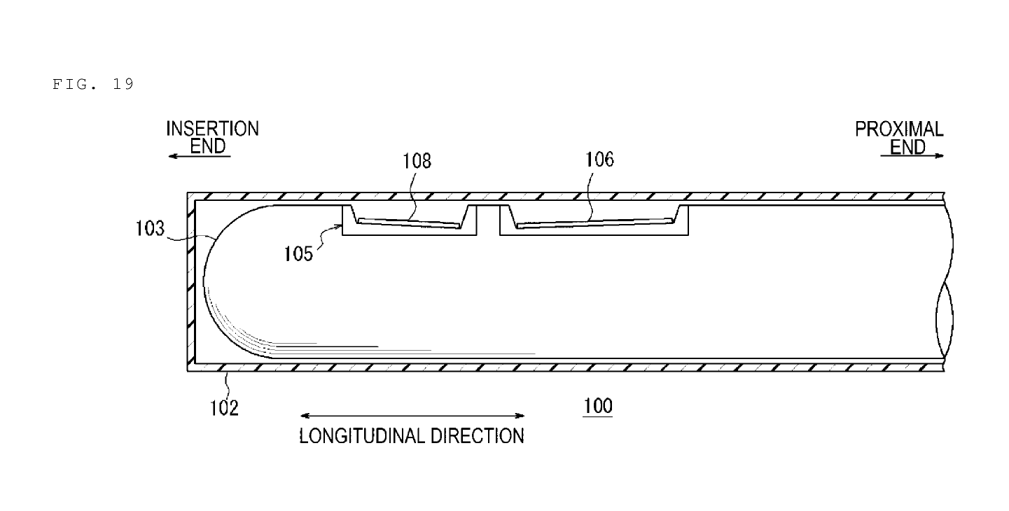

In addition, in the first to third and seventh exemplary embodiments, the first ultrasonic transducers 106 and 1065, and the imaging ultrasonic transducer 108 have approximately the same length in the longitudinal direction. However, a configuration may be made so that the length taken along the longitudinal direction of the first ultrasonic transducers 106 and 1065 is sufficiently lengthened to be longer than the length taken along the longitudinal direction of the imaging ultrasonic transducer 108 (refer to FIG. 19). The size of the first ultrasonic transducer is increased by sufficiently lengthening the first ultrasonic transducers 106 and 1065. Therefore, even without using the acoustic lens, the first ultrasonic transducer can be heated to such an extent that the biological tissues can be cauterized.

In addition, in the first to third and seventh exemplary embodiments, the first ultrasonic transducers 106 and 1065 can have a plate shape and can be configured to radiate the ultrasonic waves in a single direction, but may have a cylindrical side surface shape. This configuration can also increase the size of the first ultrasonic transducer.

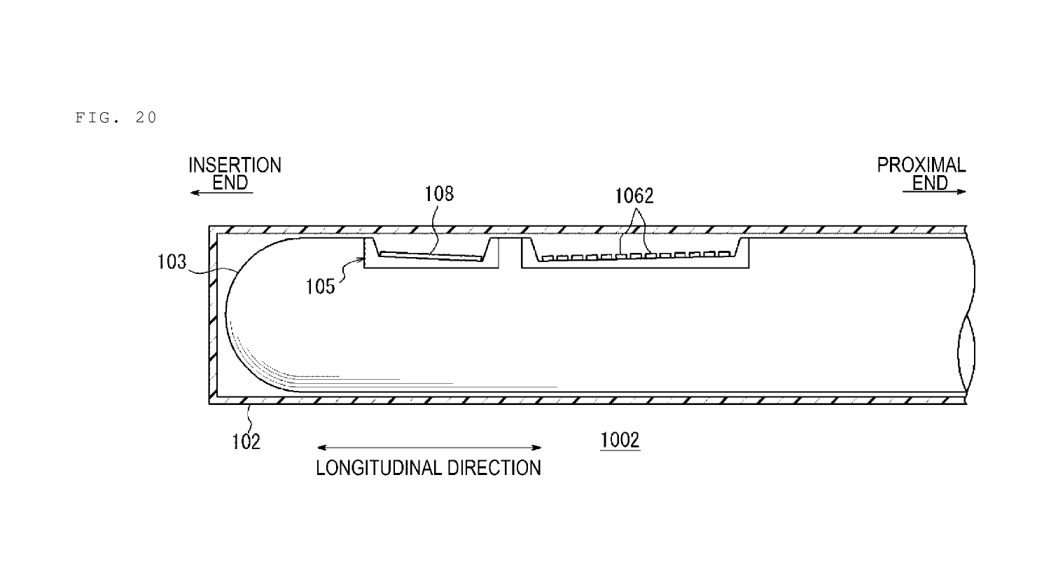

In addition, in the fourth exemplary embodiment, the length for arranging the first ultrasonic transducers 1062 side by side in the longitudinal direction can be approximately the same as the length of the imaging ultrasonic transducer. However, a configuration may be made so that the length for arranging the first ultrasonic transducers 1062 side by side is sufficiently lengthened to be longer than the length taken along the longitudinal direction of the imaging ultrasonic transducer 108 (refer to FIG. 20). The size of the first ultrasonic generator can be increased by sufficiently lengthening the length for arranging the first ultrasonic transducers 1062 side by side. Therefore, the first ultrasonic generator can be heated to such an extent that the biological tissues can be cauterized.

In addition, in the fourth exemplary embodiment, the first ultrasonic transducer 1062 can have a plate shape and can be configured to radiate the ultrasonic waves in a single direction, but may have a cylindrical side surface shape. This configuration can also increase the size of the first ultrasonic generator.

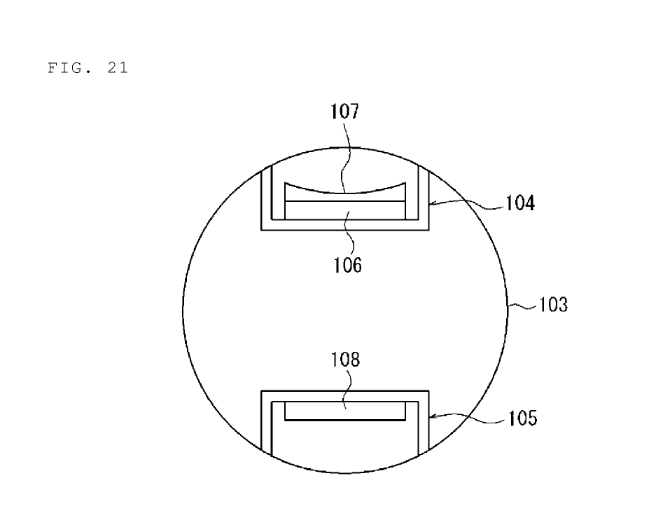

In addition, in the first, fourth, and seventh exemplary embodiments, the first ultrasonic generators 104, 1042, 1045, and the image acquisition unit 105 can be configured to be arranged side by side in the longitudinal direction of the first torque transmission body 103. However, a configuration may be made so that the first ultrasonic generator 104 and the image acquisition unit 105 are disposed at a position symmetric with respect to a plane passing through a center line taken along the longitudinal direction of the first torque transmission body 103 (refer to FIG. 21).

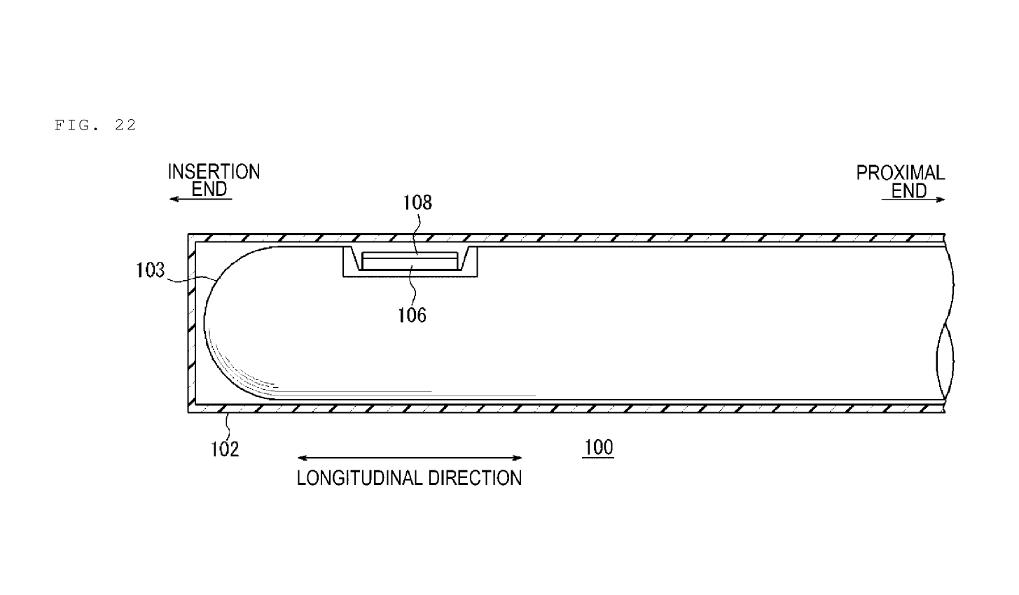

In addition, in the first embodiment, the first ultrasonic generator 104 and the imaging ultrasonic generator 105 can be configured to be separately disposed. However, a configuration may be made so that the first ultrasonic transducer 106 and the imaging ultrasonic transducer 108 are stacked on each other, as illustrated in FIG. 22. For example, a ceramic piezoelectric element can be used for the first ultrasonic transducer 106, and a piezoelectric film sheet can be used for the imaging ultrasonic transducer 108, which can form a stacked layer structure. In FIG. 22, the imaging ultrasonic transducer 108 is disposed on the sheath 102 side rather than the first ultrasonic transducer 106, but can also be reversely disposed.

In addition, the fourth exemplary embodiment has the configuration in which the first ultrasonic transducer 1062 can generate the cauterizing ultrasonic waves and the imaging ultrasonic transducer 108 can generate the imaging ultrasonic waves. However, a configuration may be made so that the first ultrasonic transducer 1062 can generate the imaging ultrasonic waves and the imaging ultrasonic transducer 108 can generate the cauterizing ultrasonic waves.

In addition, in the seventh exemplary embodiment, the first ultrasonic transducer 1065 is the single unit of the ultrasonic transducer. However, as in the fourth exemplary embodiment, a configuration may be made so that multiple ultrasonic transducers can be arranged side by side in the longitudinal direction. According to this configuration, the cauterizing ultrasonic waves can converge on the focus on a plane taken along the longitudinal direction and a plane perpendicular to the longitudinal direction.

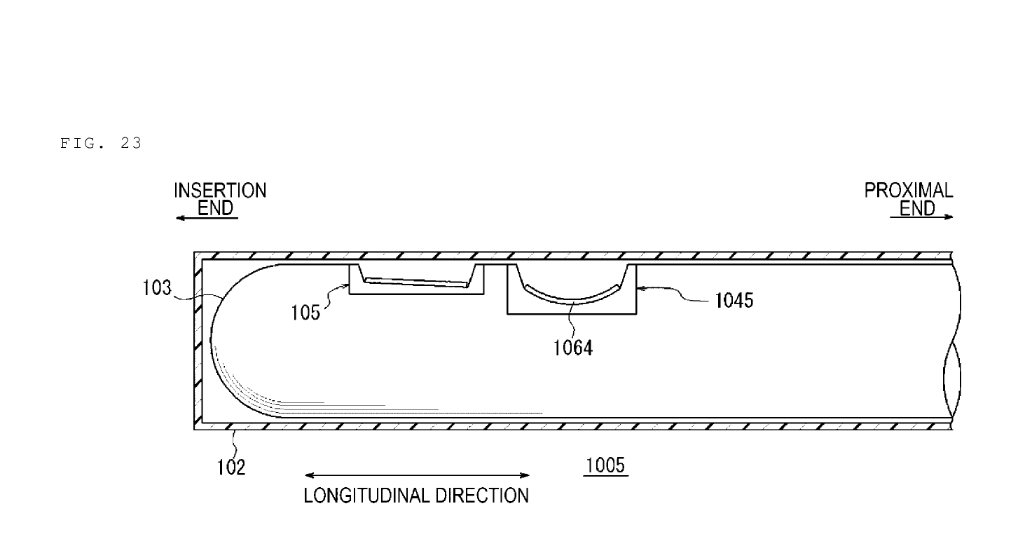

In addition, the seventh exemplary embodiment has the configuration in which the first ultrasonic transducer 1064 has the concave surface on a plane perpendicular to the longitudinal direction of the first torque transmission body 103. However, as illustrated in FIG. 23, a configuration may be made so that the first ultrasonic transducer 1064 has the concave surface along the longitudinal direction of the first torque transmission body 103.

In addition, in the first and third to sixth exemplary embodiments, the image acquisition unit 105 can be configured to acquire an image by using the ultrasonic waves. However, a configuration may be made so that the image acquisition unit 105 acquires the image, based on optical information such as TD-OCT and HUD-OCT.

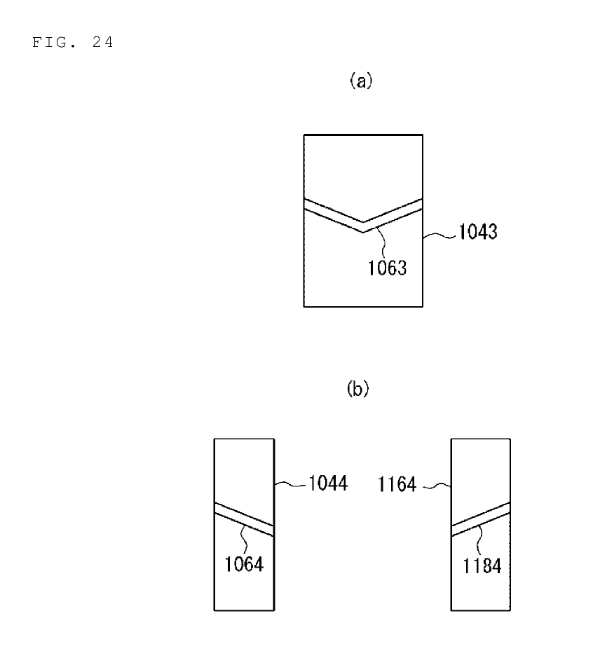

In addition, the fifth and sixth exemplary embodiments have the configuration in which the multiple ultrasonic transducers are disposed in the first ultrasonic generators 1043 and 1044, and the second ultrasonic generator 1164. However, a configuration (refer to FIG. 24) in which a single unit of the ultrasonic transducer is disposed in the respective ultrasonic generators can also obtain an effect which is the same as that of the present embodiment.

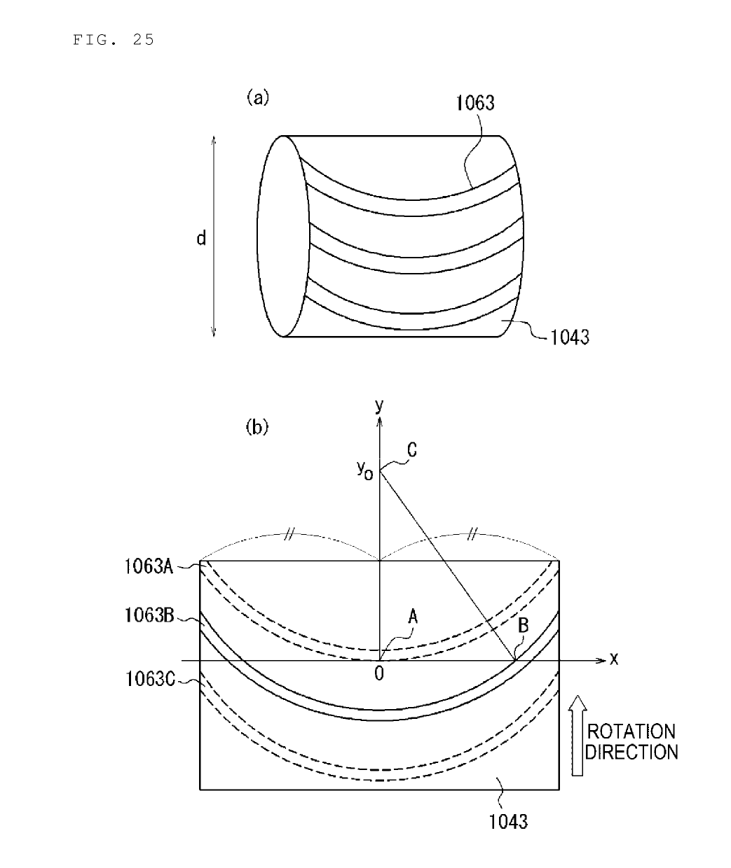

In addition, the fifth exemplary embodiment (refer to FIGS. 7 and 9) has the configuration in which the first ultrasonic transducer 1063 having the bent portion BP is disposed in the first ultrasonic generator 1043. However, without being limited thereto, for example, as illustrated in FIG. 25(a), the first ultrasonic transducer 1063 can have a smooth curvature without the bent portion BP may be disposed in the first ultrasonic generator 1043.

Here, as illustrated in FIG. 25(b), a path difference Δ1 between an ultrasonic wave radiated from a center A (x=0, y=0) of the first ultrasonic transducer 1063 toward a point C (x=0, y=yₒ) when the first ultrasonic transducer 1063 is located at a position of 1063A and an ultrasonic wave radiated from a position B (x=x, y=0) of the first ultrasonic transducer 1063 toward a point C (x=0, y=yₒ) when the first ultrasonic transducer 1063 is located at a position of 1063B is expressed by Equation (1) below.

Δl=\sqrt{x²+yₒ²}−yₒ Equation (1)

In addition, delay time τ(x) occurring due to the path difference to be corrected is expressed by Equation (2) below. Note that, C represents a speed (sound speed) of the ultrasonic wave.

$$\begin{matrix}τ ( x ) = \frac{Δ l}{C} \\ = \frac{\sqrt{{x}^{2} + {y}_{o}^{2}} - {y}_{o}}{C}\end{matrix} \qquad Equation ( 2 )$$

Furthermore, as expressed by Equation (3) below, the product of the above-described delay time τ(x) and a peripheral speed of the first ultrasonic transducer 1063 represents the function f(x) for providing a curvature of the first ultrasonic transducer 1063. Note that, a diameter of the first ultrasonic generator 1043 is d (refer to FIG. 25(a)), and a rotation speed (rps) of the first ultrasonic generator 1043 is R.

$$\begin{matrix}f ( x ) = π dR \times τ ( x ) \\ = \frac{π dR ( \sqrt{{x}^{2} + {y}_{o}^{2}} - {y}_{o} )}{C}\end{matrix} \qquad Equation ( 3 )$$

A drive frequency f of the first ultrasonic transducer 1063 is represented by 1/T, and a wavelength λ in a case of the drive frequency f of the first ultrasonic transducer 1063 is represented by CT. Note that, T is a cycle, and C is a speed (sound speed) of the ultrasonic wave.

In addition, the third exemplary embodiment has the configuration in which the first torque transmission body 1031 is formed in a tubular shape and the second torque transmission body 1091 is inserted into a lumen of the first torque transmission body 1031. However, the first torque transmission body 1031 may not be formed in the tubular shape.

The detailed description above describes blood vessel insertion-type treatment device. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A blood vessel insertion-type treatment device comprising:
a first torque transmission body having a longitudinal shape with a proximal end and a distal end, and that transmits a torque which is supplied to the proximal end of the first torque transmission body and which pivotally rotates the first torque transmission body in a longitudinal direction of the longitudinal shape;
a first ultrasonic generator disposed in the first torque transmission body, which radiates ultrasonic waves; and
wherein the first ultrasonic generator cauterizes biological tissues which are apart from the first ultrasonic generator by a predetermined distance.

2. The blood vessel insertion-type treatment device according to claim 1,
wherein the first ultrasonic generator is movable together with the first torque transmission body along the longitudinal direction.

3. The blood vessel insertion-type treatment device according to claim 1, comprising:
an image acquisition unit configured to acquire an image around the first ultrasonic generator.

4. The blood vessel insertion-type treatment device according to claim 3, comprising:
a second torque transmission body that is provided with the image acquisition unit, the second torque transmission body having a longitudinal shape with a proximal end and a distal end, and that transmits a torque which is supplied to the proximal end of the second torque transmission body and which pivotally rotates the second torque transmission body in a longitudinal direction of the longitudinal shape of the second torque transmission body.

5. The blood vessel insertion-type treatment device according to claim 4,
wherein one of the first torque transmission body and the second torque transmission body has a tubular shape, and the other one of the first torque transmission body and the second torque transmission body is inserted into a lumen of the tubular shape.

6. The blood vessel insertion-type treatment device according to claim 3,
wherein the image acquisition unit is disposed in the first torque transmission body.

7. The blood vessel insertion-type treatment device according to claim 1,
wherein the first ultrasonic generator are configured to detect reflection waves of the radiated ultrasonic waves, and configured to output the detected reflection waves as an image signal.

8. The blood vessel insertion-type treatment device according to claim 1,
wherein the first ultrasonic generator has a plurality of first ultrasonic transducers which are arranged side by side along the longitudinal direction.

9. The blood vessel insertion-type device according to claim 1, further comprising:
a pivotal rotation mechanism configured to supply a torque to the first torque transmission body, which rotates the first torque transmission body at a rotation speed corresponding to cycles of the ultrasonic waves radiated from the first ultrasonic generator.

10. The blood vessel insertion-type treatment device according to claim 9, comprising:
a first ultrasonic transducer formed in a shape having a longitudinal direction, and which is disposed in the first ultrasonic generator so as to tilt to a pivotal rotation axis formed of the first torque transmission body.

11. The blood vessel insertion-type treatment device according to claim 9,
wherein an arm portion on both sides of a bent portion of the first ultrasonic transducer having a bent shape is disposed in the first ultrasonic generator so as to tilt to a rotation axis formed of the first torque transmission body, and
wherein the pivotal rotation mechanism is configured to supply a torque to the first torque transmission body so that the first ultrasonic generator is rotated in an opening direction of the bent portion.

12. The blood vessel insertion-type treatment device according to claim 10,
wherein the first ultrasonic generator has a cylindrical side surface shape which covers a portion of a side surface of the first torque transmission body, and
wherein a plurality of first ultrasonic transducers are arranged side by side on the cylindrical side surface along a circumferential direction of the rotation performed by the first torque transmission body.

13. The blood vessel insertion-type treatment device according to claim 9, comprising:
a second ultrasonic generator that is arranged to be aligned with the first ultrasonic generator along the longitudinal direction of the first torque transmission body, and configured to radiate the ultrasonic waves for cauterizing the biological tissues which are apart from the second ultrasonic generator by a predetermined distance;
wherein a first ultrasonic transducer formed in a shape having a longitudinal direction is disposed in the first ultrasonic generator so as to tilt to a rotation axis formed of the first torque transmission body;
wherein a second ultrasonic transducer formed in a shape having a longitudinal direction is disposed in the second ultrasonic generator so as to tilt to the rotation axis formed of the first torque transmission body in a direction opposite to the first ultrasonic transducer; and
wherein the pivotal rotation mechanism supplies a torque to the first torque transmission body so that the first ultrasonic generator and the second ultrasonic generator are rotated in a direction from an intersecting point of extension lines taken along the longitudinal directions of the first ultrasonic transducer and the second ultrasonic transducer toward an intersecting point of perpendicular lines to the longitudinal directions.

14. The blood vessel insertion-type treatment device according to claim 13,
wherein the first ultrasonic generator and the second ultrasonic generator have a cylindrical side surface shape which covers a portion of a side surface of the first torque transmission body, and
wherein the multiple first ultrasonic transducers and the multiple second ultrasonic transducers are respectively arranged side by side on the cylindrical side surface of the first ultrasonic generator and the second ultrasonic generator along the circumferential direction of the rotation performed by the first torque transmission body.

15. The blood vessel insertion-type treatment device according to claim 13, comprising:
an image acquisition unit that is disposed between the first ultrasonic generator and the second ultrasonic generator, and configured to acquire an image around the first ultrasonic generator and the second ultrasonic generator.

16. The blood vessel insertion-type treatment device according to claim 1, comprising:
a tubular sheath that covers the first torque transmission body and the first ultrasonic generator; and
a balloon that is disposed near an end portion on the distal end side of the first torque transmission body of the sheath, and that expands around the circumference of the sheath.

17. The blood vessel insertion-type treatment device according to claim 16,
wherein the balloon is a cooling balloon for preventing overheating of a portion which comes into contact with the balloon when the balloon is expanded.

18. A method of cauterizing biological tissues, comprising:
inserting a blood vessel insertion-type treatment device into a blood vessel, the blood vessel insertion-type treatment device comprising:
a first torque transmission body having a longitudinal shape with a proximal end and a distal end; and
a first ultrasonic generator disposed in the first torque transmission body, which radiates ultrasonic waves;
supplying a torque to the proximal end of the first torque transmission body and which pivotally rotates the first torque transmission body in a longitudinal direction of the longitudinal shape; and
cauterizing the biological tissues which are apart from the first ultrasonic generator by a predetermined distance with the first ultrasonic generator.

19. The method according to claim 18, comprising:
providing a second torque transmission body having an image acquisition unit, the image acquisition unit configured to acquire an image around the first ultrasonic generator, and wherein the second torque transmission body has a longitudinal shape with a proximal end and a distal end; and
supplying a torque to the proximal end of the second torque transmission body and which pivotally rotates the second torque transmission body in a longitudinal direction of the longitudinal shape of the second torque transmission body.

20. The method according to claim 19,
wherein one of the first torque transmission body and the second torque transmission body has a tubular shape; and
inserting the other of the first torque transmission body and the second torque transmission body into a lumen of the one of the first torque transmission body and the second torque transmission body having the tubular shape.
